(19) United States
(12) Patent Application Publication
(10) Pub. No.: US 2012/0003321 A1
(43) Pub. Date: Jan. 5, 2012

(54) Crosslinked Dextran Composite Magnetic Microparticles and Preparation Process and Using Method Thereof

(75) Inventors: Mingli Peng, Shaanxi (CN); Yanhong Liu, Shaanxi (CN); Yali Cui, Shaanxi (CN); Chao Chen, Shaanxi (CN); Ke Li, Shaanxi (CN)

(73) Assignee: XI'AN GOLDMAG NANOBIOTECH CO. LTD., Xi'an, Shaanxi (CN)

(21) Appl. No.: 13/142,159

(22) PCT Filed: Dec. 29, 2008
(86) PCT No.: PCT/CN08/02097

§ 371 Date: Sep. 16, 2011

(30) Foreign Application Priority Data
Dec. 25, 2008 (CN) CN200810236462.8

(51) Int. Cl. A61K 9/14; A61K 31/513; A61K 31/337; A61K 31/7068; A61K 31/437; A61K 31/4375; A61K 31/704; A61K 38/14; B82Y 5/00

(52) U.S. Cl. 424/493; 514/34; 514/274; 514/449; 514/19.3; 514/49; 514/283; 977/773; 977/811; 977/906; 977/838

(57) ABSTRACT

The present invention relates to crosslinked dextran magnetic composite microparticles and a preparation process and a using method thereof. The composite microparticles comprise magnetic nanoparticles and dextran with crosslinked structure, wherein the magnetic nanoparticles are dispersed in the dextran with crosslinked structure. The process for preparing the composite microparticles comprises: preparing a dextran solution; synthesizing dextran magnetic composite microparticles; and synthesizing the crosslinked dextran magnetic composite microparticles. The using method of composite microparticles comprises: preparing crosslinked dextran magnetic composite microparticles loaded with anti-cancer drug; and adding a sustained-releasing solution thereto.

### FIELD OF THE INVENTION

The present invention relates to magnetic targeting carrier materials and a preparation process and a using method thereof, and in particular, to a crosslinked dextran magnetic composite microparticles and a preparation process and a using method thereof.

### BACKGROUND OF THE INVENTION

Magnetic polymer microspheres are multi-functional materials widely used in the field of biomedicine. Such kind of materials have not only many properties of polymer microparticles but also magnetic properties, which allow them to separate rapidly from other components upon the effect of an applied magnetic field or to orient themselves and move by magnetic field inducement. The materials have application prospects in many fields, such as, cell isolation and sorting, immunoassay, immobilized enzymes, targeting drugs, DNA isolation, nucleic acid hybridization and the like, because of the simple conditions needed in experiments, easy operation and low costs. These magnetic microparticles comprise a ferric oxide core coated with macromolecules, such as, dextran, albumin, chitosan, polyethylene glycol, cyclodextrin, polylactic acid and the like. The dextran magnetic composite microparticles have broad application prospects in the field of medicine. Dextran, also known as glucosan, is a polysaccharide with a linear main chain, formed mainly by 1,6-α-D-pyranoside linkage. As a water-soluble polysaccharide, dextran shows excellent biocompatibility and can be degraded in vivo into nonpoisonous and harmless glucose monomers. Meanwhile dextran has high reactivity, tending to react with a variety of bioactive substances, and is economically priced and easily available. Magnetic nanoparticles coated with dextran are capable of linking to bioactive substances via hydroxyl groups and keeping stable in solution via Brownian motion. At present, many reports have been made with respect to dextran magnetic composite microparticle researches: For example, Wang Guobing et al. from Huazhong University of Science and Technology have synthesized a dextran magnetic composite microparticle by virtue of a chemical coprecipitation process (Chinese Patent Application Publication No. CN101062416A); however, it can be seen from the SEM figure in the afore-mentioned patent application that the magnetic microparticles are mostly elliptical with presence of some irregularly shaped ones and have low saturation magnetization intensity, which indicates their low magnetic responsiveness. Tao, K. et al. (Colloids and Surfaces A: Physicochem. Eng. Aspects, 2006, 290, pp. 70-76) have synthesized dextran-ferroferric oxide clusters under nitrogen by virtue of coprecipitation, and also provided a theoretical structure model; however, the resulting microparticles have low magnetic responsiveness, thus limiting the application of the microparticles. Kan Zhiqiang et al. from Academy of Military Medical Sciences have also synthesized dextran magnetic microparticles by virtue of chemical coprecipitation, however, with very low saturation magnetization intensity. Xu Wei et al. from Shanghai Institute of Pharmaceutical Industry have prepared a dextran gel (Chinese Patent Application Publication No. CN1868577A). This patent application uses inorganic solid microparticles as porogen and adopts a typical oil water two-phase method: crosslinking the dextran by adding a crosslinking agent, subsequently adding an acid to dissolve the inorganic solid microparticles therein to obtain the dextran gel. The method for preparing the dextran gel in this patent application may offer a reference to present invention with respect to crosslinking dextran. Xia, Z. F. et al. (Journal of Magnetism and Magnetic Materials, 2005, 293, pp. 182-186,) have synthesized dextran-ferroferric oxide composite microparticles using ultrasound coprecipitation and presented the effect of the amounts of the dextran and the ferroferric oxide on magnetism. This paper also offers a reference to the present invention. However, the resulting magnetic particles have poor magnetism. By the already reported processes for preparing dextran magnetic composite particles, it is not possible to produce magnetic composite particles with strong magnetic response, and high drug loading capacity and capable of sustained-release.

### SUMMARY OF THE INVENTION

### Object of the Invention

To solve the technical problems existing in the background, the present invention provides a crosslinked dextran magnetic composite microparticle with high drug loading capacity and strong magnetic responsiveness and a preparation process and a using method thereof, and application thereof in targeting treatment of tumors. According to the present invention, the crosslinked dextran magnetic composite microparticles are formed by adding a crosslinking agent to dextran magnetic composite microparticles prepared by an ultrasonic method and thus crosslinking the dextran on the surface of the composite microparticles. The crosslinked dextran magnetic composite microparticles are characterized by strong magnetic responsiveness and high drug loading capacity, and being capable of highly concentrating anti-cancer drugs and can act on target cells through the positioning in a magnetic field and the sustained release of the drugs.

### Technical Solution of the Invention

Crosslinked dextran magnetic composite microparticles are characterized by comprising magnetic nanoparticles and dextran with crosslinked structure, wherein said magnetic nanoparticles are dispersed in the dextran with crosslinked structure.

The above-mentioned crosslinked dextran magnetic composite microparticles have a particle size ranging from 0.3 to 5 μm, preferably from 1 to 3 μm.

The above-mentioned magnetic nanoparticles have the composition of (Fe₂O₃)ᵣ(Fe₃O₄)₁₋ᵣ or MFe₂O₄, wherein r is 0-1 and M is Zn, Mn or Co, and have a particle size ranging from 5 to 30 nm. The dextran is a category of polysaccharides with a linear backbone formed mainly via 1,6-α-D-pyranoside linkage. It has a chemical formula of (C₆H₅O₅)ₙ and a molecular weight of 5,000-140,000, wherein the value of n depends on the molecular weight.

The process for preparing the crosslinked dextran magnetic composite microparticles is characterized by comprising the following steps:

Step 1) preparing a dextran solution, wherein

ultrapure water and an alkali solution are added to dextran to formulate a dextran solution with a concentration of 20-100 mg/ml;

Step 2) synthesizing dextran magnetic composite microparticles, wherein

magnetic nanoparticles and an alkali solution are added to the dextran solution prepared from step 1) to obtain a mixed system which is meanwhile maintained to have an alkali concentration the same as that of the dextran solution prepared from step 1); and the mixed system is stirred while reacting to synthesize the dextran magnetic composite microparticles; and

Step 3) synthesizing crosslinked dextran magnetic composite microparticles, wherein

an alkali solution is added to the dextran magnetic composite particles prepared from step 2) to obtain a mixed solution with an alkali concentration of 1-4 M; the mixed solution is fully stirred, added a crosslinking agent and subjected to reaction in water bath with stirring; and after the reaction is over, magnetical separation or centrifugation is performed to obtain the neutral crosslinked dextran magnetic composite microparticles.

The crosslinking agent in the above step 3) is added in portions, or using a constant pressure dropping funnel within no less than 1 h.

The above-mentioned crosslinking agent may be diluted with isopropanol or ethanol in a volume ratio of 1:1-1:3.

The alkali solutions in steps 1) and 2) have a concentration of 0.5-5 M. In step 1), the dissolution can be accelerated by ultrasound suitably for 2-15 min. In step 2), the magnetic nanoparticles are added in a mass ratio of 1:0.5-1:10 to the dextran from step 1), the stirring rate is 200-500 rpm; and the reaction is allowed under ultrasound at a temperature of 20-40° C. suitably for 3-8 h. In step 3), the alkali solution added has a concentration greater than that of the mixed solution so that the alkali concentration of the mixed solution can be adjusted to 1.5-3 M, the crosslinking agent is added in a mass ratio of 20:1-40:1 to the dextran, and the reaction is conducted in water bath at a stirring rate of 600-1200 rpm at 50-80° C. for 8-30 h.

The above-mentioned dextran is a category of polysaccharides with a linear backbone formed via 1,6-α-D-pyranoside linkage and having a molecular weight of 5,000-140,000. The magnetic nanoparticles have a chemical composition of (Fe₂O₃)ᵣ(Fe₃O₄)₁₋ᵣ or MFe₂O₄, and have a particle size of 5-30 nm, wherein r is 0-1, and M is Zn, Mn or Co. The magnetic nanoparticles are synthesized through methods such as chemical coprecipitation or microemulsion method, and dispersed in water or a water-miscible system. The crosslinking agent is epoxy chloropropane and the alkali solution is aqueous ammonia, or NaOH or KOH aqueous solution.

The using method of the crosslinked dextran magnetic composite microparticles is characterized in that it comprises the following steps:

Step 1) loading a drug, wherein

\textcircled{1} a suspension of the crosslinked dextran magnetic composite microparticles is added in a centrifuge tube and subjected to magnetic separation, and the supernatant is discarded;

\textcircled{2} an anticancer drug solution is added in an amount of 10-25% of that of the crosslinked dextran magnetic composite microparticles; and then the resulting solution is mixed homogenously, placed in a shaker and shaken at a constant temperature from 22-40° C. to produce crosslinked dextran magnetic composite microparticles loaded with the anticancer drug; and

\textcircled{3} the resulting crosslinked dextran magnetic composite microparticles loaded with the anticancer drug are stored at 4° C.; and

Step 2) releasing the drug, wherein

\textcircled{1} the crosslinked dextran magnetic composite microparticles loaded with the anticancer drug are added to a centrifuge tube and subjected to magnetic separation; and the supernatant is discarded; and

\textcircled{2} a sustained-releasing solution is added and the resulting mixture is shaken at a constant temperature from 36-38° C.; an appropriate amount of the sustained-releasing solution comprising the anticancer drug is removed at a specific time point; and the mixture is then replenished with a fresh sustained-releasing solution at the same amount to continue the sustained releasing of the drug.

In above step 1), the crosslinked dextran magnetic composite microparticles are used at a mass ratio of 4:1-10:1 to the anticancer drug; the shaking rate is 180-220 rpm; and the drug loading equilibrium is achieved when the concentration of the anticancer drug in the solution monitored by UV/VIS absorption spectra does not change anymore. In step 2), the crosslinked dextran magnetic composite microparticles loaded with the anticancer drug and the sustained-releasing solution have a mass ratio of 1:3 to 1:10; and the shaking is performed at a rate of 180-220 rpm at 37° C. for 7-10 days.

The anticancer drug can be Doxorubicin, daunorubicin, 5-fluorouracil, taxol, lobaplatin, bleomycin, docetaxel, gemcitabine, vinorelbine, hydroxycamptothecine and the like.

The sustained-releasing solutions can be normal saline, ultrapure water, phosphate buffer, serum, cell culture fluid or the like.

The advantages of the present invention:

1. The crosslinked dextran magnetic composite microparticles realize the sustained releasing of drugs due to the crosslinked dextran on the surface of the magnetic composite microparticles, thus being a targeting formulation having high drug loading capacity and excellent stability .

2. The crosslinked dextran magnetic composite microparticles have good magnetic responsiveness, and thus can be fixed to a particular position under a magnetic field to realize tumor targeted treatment.

3. The crosslinked dextran magnetic composite microparticles have a controllable particle size, a even particle size distribution and crosslinked structure, and thus can keep stable in the atmosphere and various solvents for a long time.

4. The process for preparing the crosslinked dextran magnetic composite microparticles is simple and costs low, and thus can be easily spread.

5. The crosslinked dextran magnetic composite microparticles can be used as a magnetic nano-carrier of a drug for tumor targeted treatment.

### DESCRIPTION OF THE DRAWINGS

FIG. 1 is a schematic diagram of the structure of the crosslinked dextran magnetic composite microparticles.

FIG. 2 is a schematic diagram of the synthetic route to the crosslinked dextran magnetic composite microparticles.

FIG. 3 is a graph of particle size distribution of the crosslinked dextran magnetic composite microparticles.

FIG. 4 is a magnetic hysteresis loop of the crosslinked dextran magnetic composite microparticles.

FIG. 5 is a graph showing the drug loading capacity of the crosslinked dextran magnetic composite microparticles versus drug loading time.

FIG. 6 a graph showing the in vitro drug releasing of the crosslinked dextran magnetic composite microparticles loaded with Doxorubicin.

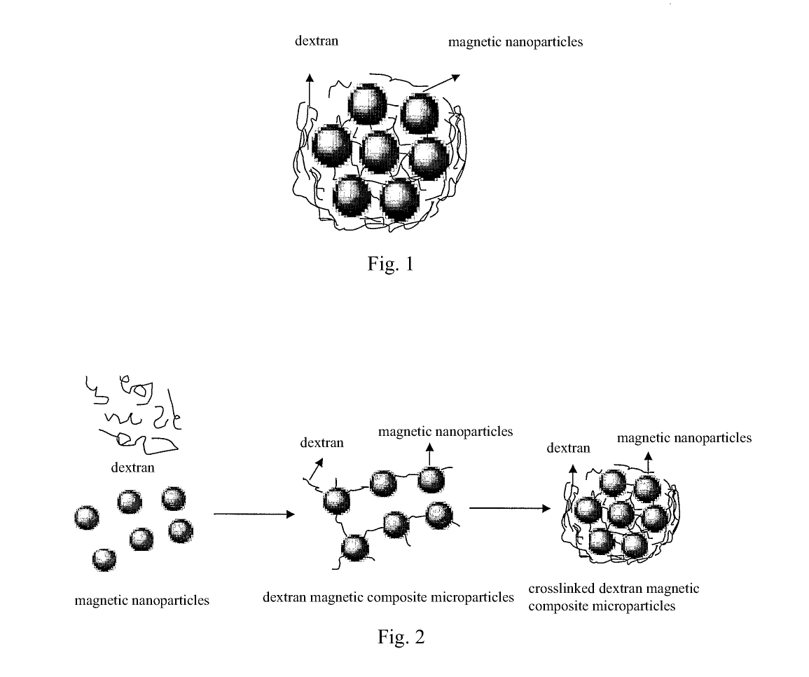

### DETAILED DESCRIPTION OF THE INVENTION

The crosslinked dextran magnetic composite microparticles according to the present invention comprise magnetic nanoparticles and dextran with crosslinked structure, wherein the magnetic nanoparticles are dispersed in the dextran with crosslinked structure.

The crosslinked dextran magnetic composite microparticles have a particle size ranging from 0.3 to 5 μm, preferably from 1 to 3 μm.

The magnetic nanoparticles have the composition of (Fe₂O₃)ᵣ(Fe₃O₄)₁₋ᵣ or MFe₂O₄, wherein r is 0-1 and M is Zn, Mn or Co, and have a particle size of 5-30 nm. The dextran is a category of polysaccharides with a linear backbone formed mainly via 1,6-α-D-pyranoside linkage. It has a chemical formula of (C₆H₅O₅)ₙ and a molecular weight of 5000-140,000, wherein the value of n depends on the molecular weight.

The process for preparing the crosslinked dextran magnetic composite comparticles involves a chemical reaction represented by:

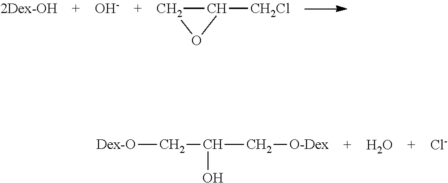

The process for preparing of the crosslinked dextran magnetic composite microparticles comprises the following steps:

Step 1) preparing a dextran solution, wherein

ultrapure water and an alkali solution are added to dextran to formulate a dextran solution with a concentration of 20-100 mg/ml;

Step 2) synthesizing dextran magnetic composite microparticles, wherein

magnetic nanoparticles and an alkali solution are added to the dextran solution prepared from step 1) to obtain a mixed system which is meanwhile maintained to have an alkali concentration the same as that of the dextran solution prepared from step 1); and the mixed system is stirred while reacting to synthesize the dextran magnetic composite microparticles; and

Step 3) synthesizing crosslinked dextran magnetic composite microparticles, wherein

an alkali solution is added to the dextran magnetic composite particles prepared from step 2) to obtain a mixed solution with an alkali concentration of 1-4 M; the mixed solution is fully stirred, added a crosslinking agent and subjected to reaction in water bath with stirring; and after the reaction is over, magnetical separation or centrifugation is performed to obtain the neutral crosslinked dextran magnetic composite microparticles.

The crosslinking agent in the above step 3) is added in portions, or using a constant pressure dropping funnel within no less than 1 h.

The above-mentioned crosslinking agent may be diluted with isopropanol or ethanol in a volume ratio of 1:1-1: 3.

The alkali solutions in steps 1) and 2) have a concentration of 0.5-5 M. In step 1), the dissolution can be accelerated by ultrasound suitably for 2-15 min. In step 2), the magnetic nanoparticles are added in a mass ratio of 1:0.5-1:10 to the dextran from step 1); the stirring rate is 200-500 rpm; and the reaction is allowed under ultrasound at a temperature of 20-40° C. suitably for 3-8 h. In step 3), the alkali solution added has a concentration greater than that of the mixed solution so that the alkali concentration of the mixed solution can be adjusted to 1.5-3 M; the crosslinking agent is added in a mass ratio of 20:1-40:1 to the dextran; and the reaction is conducted in water bath at a stirring rate of 600-1200 rpm at 50-80° C. for 8-30 h.

The above-mentioned dextran is a category of polysaccharides with a linear backbone formed via 1,6-α-D-pyranoside linkage and having a molecular weight of 5,000-140,000. The magnetic nanoparticles have a chemical composition of (Fe₂O₃)ᵣ(Fe₃O₄)₁₋ᵣ or MFe₂O₄, and have a particle size of 5-30 nm, wherein r is 0-1, and M is Zn, Mn or Co. The magnetic nanoparticles contain hydroxyl groups on the surface and can be dispersed in water or a water-miscible system. The magnetic nanoparticles are synthesized through a methods such as chemical coprecipitation or microemulsion method. The crosslinking agent is epoxy chloropropane and the alkali solution is aqueous ammonia, or NaOH or KOH aqueous solution.

The using method of the crosslinked dextran magnetic composite microparticles comprises the following steps:

Step 1) loading drug, wherein

\textcircled{1} a suspension of the crosslinked dextran magnetic composite microparticles is added in a centrifuge tube and subjected to magnetic separation, and the supernatant is discarded;

\textcircled{2} an anticancer drug solution is added in an amount of 10-25% of that of the crosslinked dextran magnetic composite microparticles; and then the resulting solution is mixed homogenously, placed in a shaker and shaken at a constant temperature from 22-40° C. to produce crosslinked dextran magnetic composite microparticles loaded with the anticancer drug; and

\textcircled{3} the resulting crosslinked dextran magnetic composite microparticles loaded with the anticancer drug are stored at 4° C.

Step 2) releasing the drug, wherein

\textcircled{1} the crosslinked dextran magnetic composite microparticles loaded with the anticancer drug are added to a centrifuge tube and subjected to magnetic separation; and the supernatant is discarded; and

\textcircled{2} a sustained-releasing solution is added and the resulting mixture is shaken at a constant temperature from 36-38° C.; an appropriate amount of the sustained-releasing solution comprising the anticancer drug is removed at a specific time point; and the mixture is then replenished with a fresh sustained-releasing solution at the same amount to continue the sustained releasing of the drug.

In above step 1), the crosslinked dextran magnetic composite microparticles are used at a mass ratio of 4:1-10:1 to the anticancer drug; the shaking rate is 180-220 rpm; and the drug loading equilibrium is achieved when the concentration of the anticancer drug in the solution monitored by UV/VIS absorption spectra does not change anymore. In step 2), the crosslinked dextran magnetic composite microparticles loaded the anticancer drug and the sustained-releasing solution have a mass ratio of 1:3 to 1:10; and the shaking is performed at a rate of 180-220 rpm at 37° C. suitably for 7-10 days.

The anticancer drug can be Doxorubicin, daunorubicin, 5-fluorouracil, taxol, lobaplatin, bleomycin, docetaxel, gemcitabine, vinorelbine, hydroxycamptothecine and the like.

The sustained-releasing solutions can be normal saline, ultrapure water, phosphate buffer, serum, cell culture fluid or the like.

Examples of the process for preparing the crosslinked dextran magnetic composite microparticles are as follows:

### EXAMPLE 1

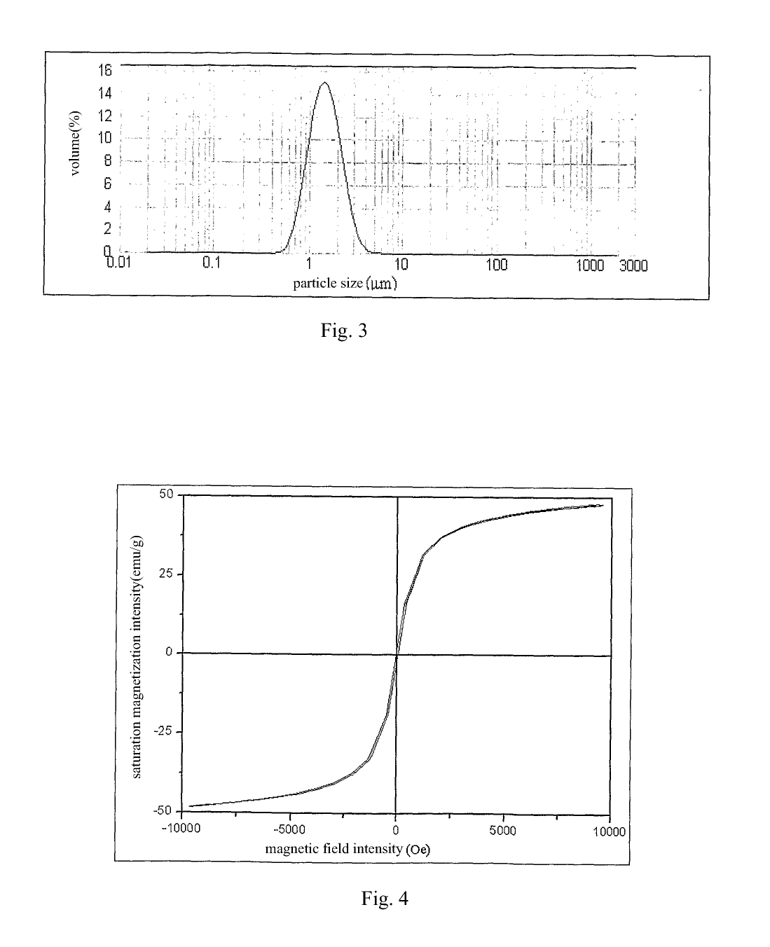

2 g Dextran-40, followed by 10 ml ultrapure water and 10 ml 1M NaOH, were added to a 250 ml round bottom flask to undergo ultrasound dissolution. Then, 400 mg magnetic nanoparticles (the solid content is about 20 mg/ml) and an equal volume of 1M NaOH were added to the 250 ml round bottom flask to react under ultrasound with stirring at a controlled rate of 400 rpm at 26° C. for 4 h. NaOH was added to adjust the alkali concentration of the system to 3 M, and then 24 ml epoxy chloropropane diluted in isopropanol at a ratio of 1:1 was added by a constant pressure dropping funnel within 1 h. The resulting system was then heated to 60° C. and continued to react with stirring at a controlled rate of 900 rpm for 12 h. After the reaction was over, magnetic separation, centrifugation, or the like was carried out to make the system be neutral and to obtain crosslinked dextran magnetic composite microparticles. They had a particle size of about 1-3 μm, detected by a laser scattering particle size analyzer (see FIG. 3), and saturation magnetization intensity of more than 40 emu/g (see, FIG. 4).

### EXAMPLE 2

10 ml Ultrapure water and 10 ml of 1.3 M NaOH, followed by 2 g dextran-20, were added to a 250 ml round bottom flask to undergo ultrasound dissolution. Then 400 mg magnetic nanoparticles (the solid content is about 20 mg/ml) and an equal volume of NaOH (1.3 M) were added to the 250 ml round bottom flask to react under ultrasound at a controlled stirring rate of 300 rpm at 28° C. for 6 h. NaOH was added to adjust the alkali concentration of the system to 2 M, and then 48 ml epoxy chloropropane diluted in isopropanol at a ratio of 1:2 was added by a constant pressure dropping funnel within 1.5 h. The resulting system was then heated to 70° C. and continued to react with stirring at a controlled rate of 1,200 rpm for 28 h. After the reaction was over, magnetic separation, centrifugation, or the like was carried out to make the system be neutral and to obtain crosslinked dextran magnetic composite microparticles. The resulting crosslinked dextran magnetic composite microparticles had a particle size of about 1-3 μm, detected by a laser scattering particle size analyzer (see FIG. 3), and saturation magnetization intensity of more than 40 emu/g (see, FIG. 4).

### EXAMPLE 3

2 g Dextran-30, followed by 10 ml ultrapure water and 10 ml of 1M NaOH, were added to a 250 ml round bottom flask to undergo ultrasound dissolution. Then, 400 mg magnetic nanoparticles (the solid content is about 20 mg/ml) and an equal volume of 1M NaOH were added to the 250 ml round bottom flask to react under ultrasound with stirring at a controlled rate of 500 rpm at 27° C. for 8 h. NaOH was added to adjust the alkali concentration of the system to 3 M, and then 60 ml epoxy chloropropane diluted in ethanol at ratio of 1:3 was added in three even portions. The resulting system was then heated to 60° C. and continued to react with stirring at a controlled rate of 1,000 rpm for 20 h. After the reaction was over, magnetic separation, centrifugation or the like was carried out to make the system be neutral and to obtain crosslinked dextran magnetic composite microparticles. They had a particle size of about 1-3 μm, detected by a laser scattering particle size analyzer (see FIG. 3), and saturation magnetization intensity of more than 40 emu/g (see, FIG. 4).

### EXAMPLE 4

2 g Dextran-70, followed by 10 ml ultrapure water and 10 ml NaOH (1.5 M) were added to a 250 round bottom flask to undergo ultrasound dissolution. Then, 400 mg magnetic nanoparticles (the solid content is about 20 mg/ml) and an equal volume of NaOH (1.5 M) were added to the 250 round bottom flask to react under ultrasound with stirring at a controlled rate of 300 rpm at 25° C. for 8 h. NaOH was added to adjust the alkali concentration of the system to 1.5 M, then 40 ml epoxy chloropropane diluted in ethanol at a ratio of 1:1 was added in three even portions. The resulting system was then heated to 60° C. and continued to react with stirring at a controlled rate of 1,000 rpm for 15 h. After the reaction was over, magnetic separation, centrifugation or the like was carried out to make the system be neutral and to obtain crosslinked dextran magnetic composite microparticles. The resulting crosslinked dextran magnetic composite microparticles had a particle size of about 1-3 μm, detected by a laser scattering particle size analyzer (see FIG. 3), and saturation magnetization intensity of more than 40 emu/g (see, FIG. 4).

### EXAMPLE 5

2 g Dextran-40, followed by 10 ml ultrapure water and 10 ml aqueous ammonia, were added to a 250 ml round bottom flask to undergo ultrasound dissolution. Then, 400 mg magnetic nanoparticles (the solid content is about 20 mg/ml) and an equal volume of aqueous ammonia were added to the 250 ml round bottom flask to react under ultrasound with stirring at a controlled rate of 400 rpm at 25° C. for 6 h. Aqueous ammonia was added to adjust the alkali concentration of the system to 1.5 M, then 36 ml epoxy chloropropane was added in two even portions. The resulting system was then heated to 60° C. and continued to react with stirring at a controlled rate of 1,000 rpm for 20 h. After the reaction was over, magnetic separation, centrifugation or the like was carried out to make the system be neutral and to obtain crosslinked dextran magnetic composite microparticles. They have a particle size of about 1-3 μm, detected by a laser scattering particle size analyzer (see FIG. 3), and saturation magnetization intensity of more than 40 emu/g (see, FIG. 4).

The using method of the crosslinked dextran magnetic composite microparticles according to the present invention is now exemplified by doxorubicin as the used drug.

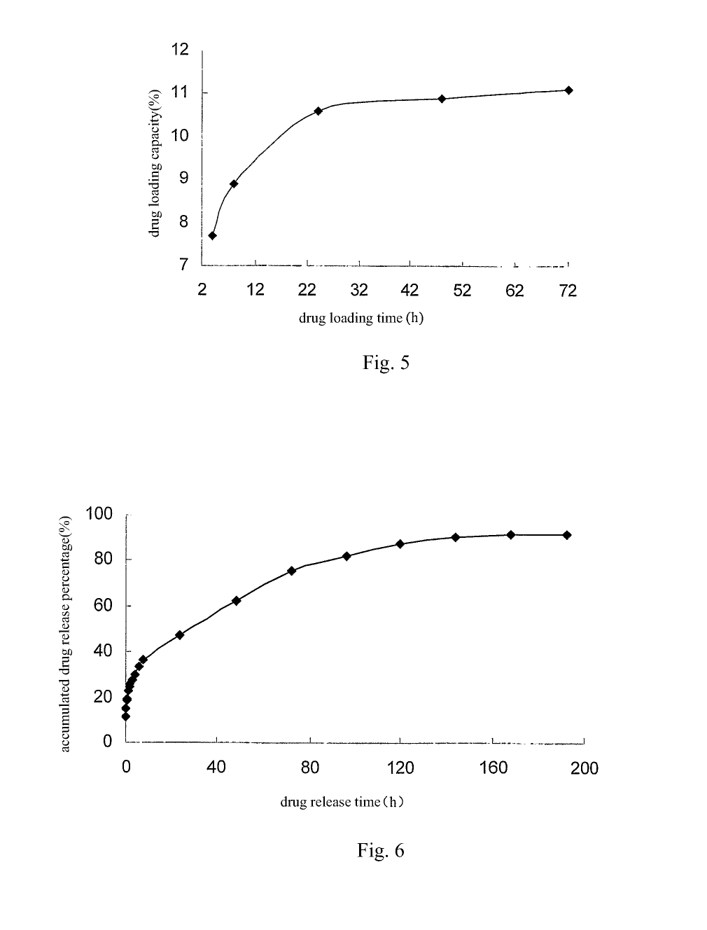

5 mg of the crosslinked dextran magnetic composite microparticles (the solid content was 15 mg/ml) was added in a 5 ml centrifuge tube and subjected to magnetic separation. The supernatant was discarded. 1 ml Doxorubicin solution (1 mg/ml) was added. The resulting system was shaken with a rate of 180 rpm at 25° C. for 72 h to achieve the drug loading equilibrium, and stored at 4° C. The resulting product was the crosslinked dextran magnetic composite microparticles loaded with doxorubicin. The residual doxorubicin in the solution was determined by a ultraviolet spectrophotometer and the drug loading capacity and the encapsulation ratio were calculated by the two following equations respectively. The drug loading capacity was 11% (see FIG. 5) and the encapsulation ratio was 82%.

$$drug loading capacity = \frac{total amount of Dox - amount of free Dox}{\begin{matrix}total amount of the complex \\ magnetic particles loaded with drug\end{matrix}} \times 100 \%$$

$$encapsulation ratio = \frac{total amount of Dox - amount of free Dox}{total amount of Dox} \times 100 \%$$

The crosslinked dextran magnetic composite microparticles loaded with doxorubicin were added to a 50 ml centrifuge tube comprising 15 ml PBS at pH 7.4. Then, the tube was placed in a constant temperature shaker to be shaken at a rate of 180 rpm at 37° C. for 8 days. 0.5 ml of the sustained-releasing solution comprising doxorubicin was removed at a specific time point. And 0.5 ml fresh PBS buffer was added as replenishment. The amount of the accumulated doxorubicin released into the PBS was determined by a fluorescence spectrophotometer and the accumulated drug release percentage was 91%. The releasing effect was good with no occurrence of burst release, see, FIG. 6. The accumulated drug release percentage was calculated by the following equation:

$$acccumulated drug release percentage ( \% ) = \frac{{\sum }_{n - 1}^{i} {C}_{i} {V}_{i} + {C}_{n} V}{W \cdot D} \times 100 \%$$

wherein Ci, and Cn are the drug concentration of the releasing medium, Vi is the volume of the removed releasing medium, V is the total volume of the releasing medium, W is the weight of the microparticles and D is drug content of the microparticles.

1. Crosslinked dextran magnetic composite microparticles, characterized by comprising magnetic nanoparticles and dextran with crosslinked structure, wherein said magnetic nanoparticles are dispersed in the dextran with crosslinked structure.

2. The crosslinked dextran magnetic composite microparticles according to claim 1, characterized by having a particle size ranging from 0.3 to 5 μm.

3. The crosslinked dextran magnetic composite microparticles according to claim 2, characterized by having a particle size ranging from 1 to 3 μm.

4. The crosslinked dextran magnetic composite microparticles according to claim 1, characterized in that: said magnetic nanoparticles have the composition of (Fe₂O₃)ᵣ(Fe₃O₄)₁₋ᵣ or MFe₂O₄, wherein r is 0-1 and M is Zn, Mn or Co, and have a particle size ranging from 5 to 30 nm; and said dextran is a category of polysaccharides with a linear backbone formed mainly via 1,6-α-D-pyranoside linkage, and having a chemical formula of (C₆H₅O₅)ₙ and a molecular weight of 5,000-140,000.

5. A process for preparing the crosslinked dextran magnetic composite microparticles according to claim 1, characterized by comprising the following steps:
Step 1) preparing a dextran solution, wherein
ultrapure water and an alkali solution are added to dextran to formulate a dextran solution with a concentration of 20-100 mg/ml;
Step 2) synthesizing dextran magnetic composite microparticles, wherein
to the dextran solution prepared from step 1), a magnetic nanoparticle solution and an alkali solution are added to obtain a mixed system which is meanwhile maintained to have an alkali concentration the same as that of the dextran solution prepared from step 1); and the mixed system is stirred while reacting to synthesize the dextran magnetic composite microparticles; and
Step 3) synthesizing crosslinked dextran magnetic composite microparticles, wherein
to the dextran magnetic composite particles prepared from step 2), an alkali solution is added to obtain a mixed solution with an alkali concentration of 1-4 M; the mixed solution is fully stirred, added a crosslinking agent and subjected to reaction in water bath with stirring; and after the reaction is over, magnetical separation or centrifugation is performed obtain the neutral crosslinked dextran magnetic composite microparticles.

6. A process for preparing the crosslinked dextran magnetic composite microparticles according to claim 5, characterized in that the crosslinking agent in step 3) is added in portions, or using a constant pressure dropping funnel within no less than 1 h.

7. A process for preparing the crosslinked dextran magnetic composite microparticles according to claim 5, characterized in that said crosslinking agent is diluted with isopropanol or ethanol in a volume ratio of 1:1-1:3.

8. A process for preparing the crosslinked dextran magnetic composite microparticles according to claim 7, characterized in that: the alkali solutions in steps 1) and 2) have a concentration of 0.5-5 M; in step 1), the dissolution can be accelerated by ultrasound suitably for 2-15 min; in step 2), the magnetic nanoparticles are added in a mass ratio of 1:0.5-1:10 to the dextran from step 1), the stirring rate is 200-500 rpm, and the reaction is allowed under ultrasound at a temperature of 20-40° C. suitably for 3-8 h; and in step 3), the alkali solution added has a concentration greater than that of the mixed solution so that the alkali concentration of the mixed solution can be adjusted to 1.5-3 M, the crosslinking agent is added in a mass ratio of 20:1-40:1 to the dextran, and the reaction is conducted in water bath at a stirring rate of 600-1200 rpm at 50-80° C. for 8-30 h.

9. A process for preparing the crosslinked dextran magnetic composite microparticles according to claim 8, characterized in that: said dextran is a category of polysaccharides with a linear backbone formed mainly via 1,6-α-D-pyranoside linkage and having a molecular weight of 5,000-140,000; said magnetic nanoparticles have the chemical composition of (Fe₂O₃)ᵣ(Fe₃O₄)₁₋ᵣ or MFe₂O₄, wherein r is 0-1, and M is Zn, Mn or Co, have the particle size of 5-30 nm, comprise hydroxyl groups on the surfaces, thus capable of dispersing in water or a water-miscible system, and are synthesized through a method such as a chemical coprecipitation or microemulsion method; the crosslinking agent is epoxy chloropropane; and the alkali solution is aqueous ammonia, NaOH or KOH aqueous solution.

10. A using method of the crosslinked dextran magnetic composite microparticles according to claim 1, characterized by comprising the following steps:
Step 1) loading drug, wherein
\textcircled{1} a suspension of the crosslinked dextran magnetic composite microparticles is added in a centrifuge tube and subjected to magnetic separation, and the supernatant is discarded;
\textcircled{2} an anticancer drug solution is added in an amount of 10-25% of that of the crosslinked dextran magnetic composite microparticles; and then the resulting solution is mixed homogenously, placed in a shaker and shaken at a constant temperature from 22-40° C. to produce crosslinked dextran magnetic composite microparticles loaded with the anticancer drug; and
\textcircled{3} the resulting crosslinked dextran magnetic composite microparticles loaded with the anticancer drug are stored at 4° C.;
Step 2) releasing drug, wherein
\textcircled{1} the crosslinked dextran magnetic composite microparticles loaded with the anticancer drug are added to a centrifuge tube and subjected to magnetic separation; and the supernatant is discarded; and
\textcircled{2} a sustained-releasing solution is added and the resulting mixture is shaken at a constant temperature from 36-38° C., an appropriate amount of the sustained-releasing solution comprising the anticancer drug is removed at a specific time point; and the mixture is then replenished with a fresh sustained-releasing solution at the same amount to continue the sustained releasing of the drug.

11. The using method of the crosslinked dextran magnetic composite microparticles according to claim 10, characterized in that: in above step 1), the crosslinked dextran magnetic composite microparticles are used at a mass ratio of 4:1-10:1 to the anticancer drug, the shaking rate is 180-220 rpm, and the drug loading equilibrium is achieved when the concentration of the anticancer drug in the solution monitored by UV/VIS absorption spectra does not change anymore; and in step 2), the crosslinked dextran magnetic composite microparticles loaded with the anticancer drug and the sustained-releasing solution have a mass ratio of 1:3 to 1:10, and the shaking is performed at a rate of 180-220 rpm at 37° C. for 7-10 days.

12. The using method of the crosslinked dextran magnetic composite microparticles according to claim 11, characterized in that said anticancer drug is selected from doxorubicin, daunomycin, 5-fluorouracil, taxol, lobaplatin, bleomycin, docetaxel, gemcitabine, vinorelbine and hydroxycamptothecine.

13. The using method of the crosslinked dextran magnetic composite microparticles according to claim 12, characterized in that said sustained-releasing solutions is normal saline, ultrapure water, phosphate buffer, serum or cell culture fluid.

14. The crosslinked dextran magnetic composite microparticles according to claim 2, characterized in that: said magnetic nanoparticles have the composition of (Fe₂O₃)ᵣ(Fe₃O₄)₁₋ᵣ or MFe₂O₄, wherein r is 0-1 and M is Zn, Mn or Co, and have a particle size ranging from 5 to 30 nm; and said dextran is category of polysaccharides with a linear backbone formed mainly via 1,6-α-D-pyranoside linkage, and having a chemical formula of (C₆H₅O₅)ₙ and a molecular weight of 5,000-140,000.

15. The crosslinked dextran magnetic composite microparticles according to claim 3, characterized in that: said magnetic nanoparticles have the composition of (Fe₂O₃)ᵣ(Fe₃O₄)₁₋ᵣ or MFe₂O₄, wherein r is 0-1 and M is Zn, Mn or Co, and have a particle size ranging from 5 to 30 nm; and said dextran is category of polysaccharides with a linear backbone formed mainly via 1,6-α-D-pyranoside linkage, and having a chemical formula of (C₆H₅O₅)ₙ and a molecular weight of 5,000-140,000.

16. A process for preparing the crosslinked dextran magnetic composite microparticles according to claim 6, characterized in that said crosslinking agent is diluted with isopropanol or ethanol in a volume ratio of 1:1-1:3.

17. A process for preparing the crosslinked dextran magnetic composite microparticles according to claim 16, characterized in that: the alkali solutions in steps 1) and 2) have a concentration of 0.5-5 M; in step 1), the dissolution can be accelerated by ultrasound suitably for 2-15 min; in step 2), the magnetic nanoparticles are added in a mass ratio of 1:0.5-1:10 to the dextran from step 1), the stirring rate is 200-500 rpm, and the reaction is allowed under ultrasound at a temperature of 20-40° C. suitably for 3-8 h; and in step 3), the alkali solution added has a concentration greater than that of the mixed solution so that the alkali concentration of the mixed solution can be adjusted to 1.5-3 M, the crosslinking agent is added in a mass ratio of 20:1-40:1 to the dextran, and the reaction is conducted in water bath at a stirring rate of 600-1200 rpm at 50-80° C. for 8-30 h.

18. A process for preparing the crosslinked dextran magnetic composite microparticles according to claim 17, characterized in that: said dextran is a category of polysaccharides with a linear backbone formed mainly via 1,6-α-D-pyranoside linkage and having a molecular weight of 5,000-140,000; said magnetic nanoparticles have the chemical composition of (Fe₂O₃)ᵣ(Fe₃O₄)₁₋ᵣ or MFe₂O₄, wherein r is 0-1, and M is Zn, Mn or Co, have the particle size of 5-30 nm, comprise hydroxyl groups on the surfaces, thus capable of dispersing in water or a water-miscible system, and are synthesized through a method such as a chemical coprecipitation or microemulsion method; the crosslinking agent is epoxy chloropropane; and the alkali solution is aqueous ammonia, NaOH or KOH aqueous solution.
